(19) United States
(12) Patent Application Publication
(10) Pub. No.: US 2009/0264966 A1
(43) Pub. Date: Oct. 22, 2009

(54) Device for Inductive Charging of Implanted Electronic Devices

(75) Inventors: Ronald D. Blum, Roanoke, VA (US); Joshua N. Haddock, Roanoke, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PixelOptics, Inc., Roanoke, VA (US)

(21) Appl. No.: 12/465,970

(22) Filed: May 14, 2009

Related U.S. Application Data
(63) Continuation-in-part of application No. 11/261,035, filed on Oct. 28, 2005.
(60) Provisional application No. 60/623,946, filed on Nov. 2, 2004.
(60) Provisional application No. 60/636,490, filed on Dec. 17, 2004.
(60) Provisional application No. 61/053,796, filed on May 16, 2008.

(51) Int. Cl. A61N 1/02

(52) U.S. Cl. 607/61

(57) ABSTRACT

Devices suitable for charging implanted electronic devices are provided. A device suitable for charging one or more implanted electronic devices, specifically implanted ophthalmic devices, may include a wearable frame, one or more conductive coils, and a power source to provide a current to the conductive coil. When placed in proximity to an implanted device having a second conductive coil, the current in the conductive coil causes an induced current in the second conductive coil, which may be used to power the implanted electronic device.

### CROSS-REFERENCES TO RELATED APPLICATIONS

This application claims priority to U.S. Provisional Application No. 61/053,796, filed May 16, 2008, the disclosure of which is incorporated by reference in its entirety. This application is a continuation-in-part of U.S. application Ser. No. 11/261,035, filed Oct. 28, 2005, which claims priority to U.S. Provisional Application No. 60/623,946, filed Nov. 2, 2004, and U.S. Provisional Application No. 60/636,490, filed Dec. 17, 2004, the disclosure of each of which is incorporated by reference in its entirety.

### BACKGROUND OF THE INVENTION

Various implantable ophthalmic devices have been proposed to cure illness or injury, correct optical errors of the eye, enhance vision, and achieve other objectives. These devices may be implanted into the cornea (corneal inlays), as replacements for the crystalline lens (Intra-Ocular lenses, IOLs), in front of or behind a healthy crystalline lens in the anterior or posterior chamber, onto the retina (synthetic retina), under the retina, or in other areas of the eye. By way of example only, examples of implantable electronic devices are provided in U.S. patent application Ser. No. 12/039,779, filed Feb. 22, 2008, and U.S. application Ser. No. 12/406,656, filed Mar. 18, 2009, the disclosure of each of which is incorporated by reference in its entirety. Electronic devices have also been proposed or implemented which are implanted within the brain, ear, and in other regions of the face or frontal area of the head.

### BRIEF SUMMARY OF THE INVENTION

A device suitable for charging one or more implanted electronic devices, specifically implanted ophthalmic devices, may include a wearable frame, one or more conductive coils, and a power source to provide a current to the conductive coil. When placed in proximity to an implanted device having a second conductive coil, the current in the conductive coil causes an induced current in the second conductive coil, which may be used to power the implanted electronic device.

### BRIEF DESCRIPTION OF THE DRAWINGS

FIG. 1 shows an example schematic view of a coil placed in proximity to an implanted medical device.

FIG. 2A shows a schematic diagram of an example device suitable for charging an implanted ophthalmic device.

FIG. 2B shows a schematic diagram of an example device suitable for charging one or more implanted ophthalmic devices.

FIG. 3A shows a schematic front view of an example device suitable for charging one or more implanted ophthalmic devices.

FIG. 3B shows a schematic top view of an example device suitable for charging one or more implanted ophthalmic devices.

FIG. 4A shows a schematic front view of an example device suitable for charging one or more implanted ophthalmic devices.

FIG. 4B shows a schematic top view of an example device suitable for charging one or more implanted ophthalmic devices.

### DETAILED DESCRIPTION OF THE INVENTION

Various implantable electronic devices, including implantable ophthalmic devices, may include or are anticipated to include an integrated electrical power source, such as a battery. Some implantable devices may be suitable for use with an integrated, rechargeable battery. For such devices, a non-invasive technique of recharging the power source may be preferred, as replacing the power source would likely require additional invasive surgical procedures.

A rechargeable implantable device, i.e., a device incorporating a rechargeable battery or other rechargeable energy storage mechanism, may be charged by inducing a current in the device. As would be known to one of skill in the art, an alternating current (AC) applied to a first conductive coil causes an alternating current in a second coil that is positioned near, but not in direct physical contact with, the first coil via Faraday's law of induction. The changing current in the first coil causes a magnetic field in the region of the coils. As the current changes, so does the magnetic field, causing an induced current in the second coil. This induced current may be used to charge a battery or other energy storage mechanism in electrical contact with the coil. The amplitude of the induced current may be controlled by adjusting the current applied to the first coil and the relative positioning of the two coils. In general, the transfer of energy into the second coil and, therefore, into the rechargeable battery will be more efficient as the central axes of the coil are more closely aligned.

One method previously contemplated by the inventors for charging an implanted device is to use an inductive pillow. An example of an inductive pillow is provided in U.S. application Ser. No. 11/261,035, filed Oct. 28, 2005, the disclosure of which is incorporated by reference in its entirety. Such a device incorporates a charging coil into a pillow on which the user may sleep. A power source within or connected to the pillow provides current to the charging coil, which inductively charges an implanted device. However, the pillow may not be as efficient as desired, since a user typically will turn from side to side or will roll over on to their back when sleeping. Thus, in many circumstances the inductive pillow may not properly or completely charge the device, or the pillow may provide an inductive charge, but the efficiency of the inductive transfer will be reduced.

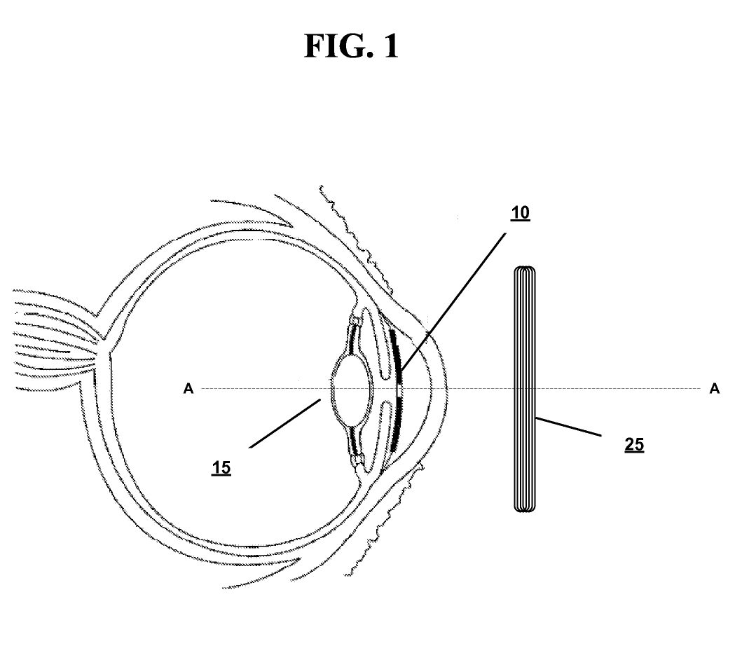

Devices as described herein may provide more efficient charging and may provide other benefits relative to other methods, such as an inductive pillow. To charge a battery of an implanted medical device, a coil may be placed in proximity to the implanted device and a current provided to the coil. A second current thus may be induced in a coil within the implanted device, charging a battery electrically connected to the coil. FIG. 1 shows an example schematic view of a coil placed in proximity to an implanted medical device. A wearer may have one or more implanted devices, such as an intra-ocular optic (100) 10, an intra-ocular lens (IOL) 15, a combination thereof, or one or more other devices. An implanted device 10, 15 may include a rechargeable battery electrically connected to a conductive coil. A conductive coil 25 may be placed in proximity to the devices, and a current applied to the coil 25. A second current may be generated in the coil in the device 10, 15, thus charging the rechargeable battery. As will be described in further detail below, the conductive coil 25 may be incorporated into a variety of external headwear or other devices that may be used to position the coil in an appropriate location on the wearer's head.

In some configurations, it may be preferred for the conductive coil 25 to be placed in a specific configuration or alignment relative to the implanted device. For example, an IOL 15 or other implanted ophthalmic device may be known to have a certain arrangement relative to the wearer's eye, and may incorporate a coil in a known arrangement. In an example arrangement, the coil incorporated into the device 15 may be arranged so that the central axis of the coil is parallel or approximately parallel to a line drawn through the center or along the major axis of the wearer's eye, such as line A in FIG. 1. This particular arrangement is provided only as an example, and it will be understood that other the coil 25 may be disposed in various other arrangements and configurations. If the arrangement of the device coil is known, the external conductive coil 25 may be positioned such that the coil 25 is arranged to maximize or achieve a desired efficiency of the charging process. For example, a specific arrangement may produce the maximum induced current in the device coil that can be obtained for a particular combination of coil size current applied to the coil 25. Although it may be preferred for a coil within the device 10, 15 to be placed in a certain alignment to the coil 25, other configurations may be used. For example, in some configurations the exact arrangement or positioning of a coil within the implanted device may not be known. As will be apparent to one of skill in the art, the size of the coil 25 and magnitude of the current applied to the coil 25 may be adjusted to achieve a desired induced current within the device. Similarly, the charging time may be adjusted to achieve a desired total charge applied to the rechargeable battery.

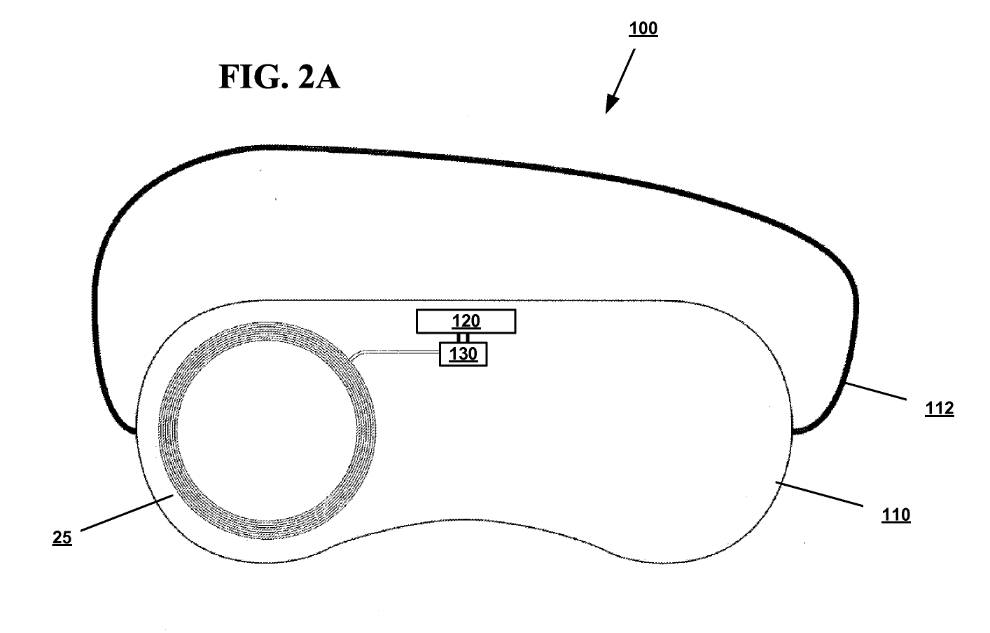

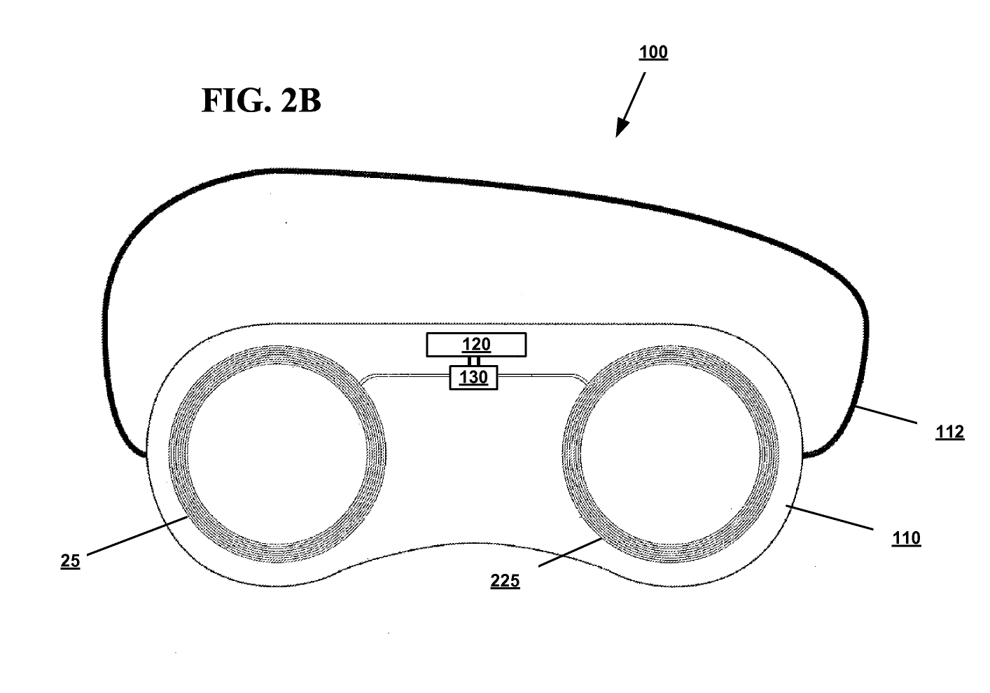

FIGS. 2A and 2B show cut-away schematic diagrams of example devices suitable for placing a charging coil in proximity to an implanted device, such as an implanted ophthalmic device having a rechargeable battery. A device may include a wearable frame 100 such as the mask illustrated in FIGS. 2A and 2B. The wearable frame may include a portion for removeably or temporarily attaching the frame 100 to the body of a wearer, such as an elastic or adjustable strap 112 suitable for attaching the frame 100 to the wearer's head. Other structures may be used, such as frames used with sunglasses, prescription lenses, reading glasses, and the like. The frame 100 may include a central or main portion 110 that is placed adjacent to a desired region of the wearer's body. The device may include one or more conductive coils 25, 225 which, when the device is worn, may be disposed adjacent to a region of the wearer's body in which an implantable electronic device is implanted. A power source 120, such as a battery, may provide power to generate a current in the conductive coils 25, 225. The device may include various control electronics 130, such as to convert power provided by the power source 120 into an alternating current having a desired frequency, amplitude, or other characteristics, as will be understood by one of skill in the art.

As specific examples, the devices shown in FIGS. 2A and 2B include a mask 110, which may be similar in design to masks currently available for blocking light while sleeping in bright lighting. The mask may be placed over the eyes of a wearer and held in place by an elasticized strap 112 placed behind the wearer's head. A battery 120 may be either disposable or rechargeable. Recharging the battery may be accomplished by, for example, plugging the mask 100 or the battery 120 into a charging unit similar to those used for mobile phones or other portable electronics. The battery provides electrical power via power leads to drive electronics 130 which supply the charging coils with an AC current sufficient to induce an AC current in a second coil within an implanted device, such as an implanted electronic ophthalmic device. The AC current induced in the second coil is then used to power recharging circuitry within the implanted ophthalmic device.

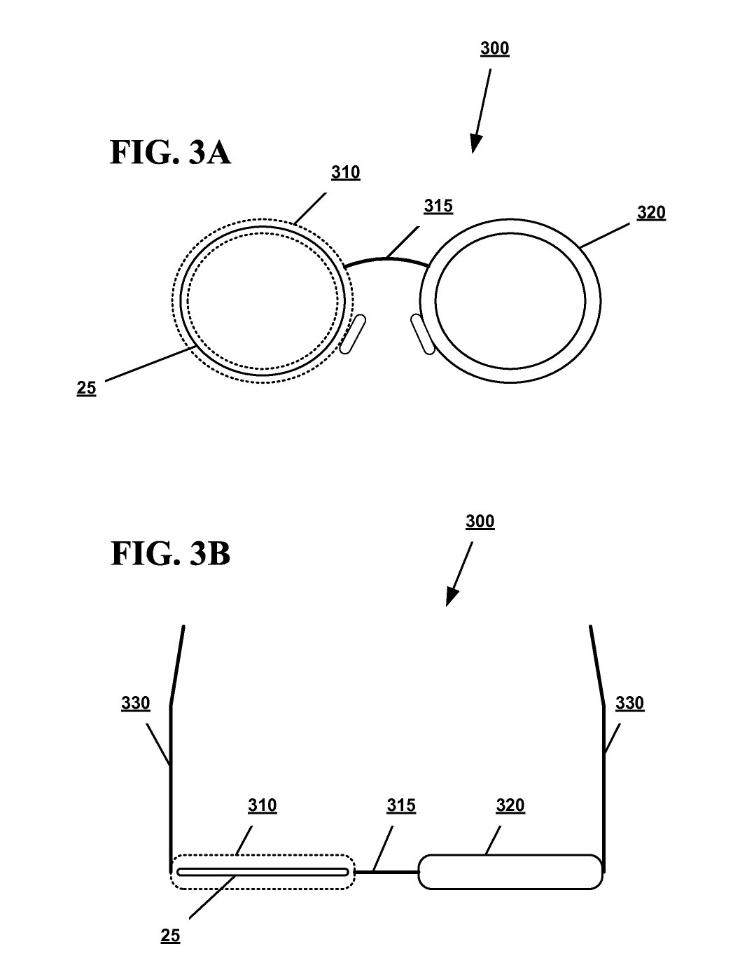

Various configurations may be used for the wearable frame. For example, FIGS. 3A and 3B show front and top schematic views, respectively, of a device 300 that includes a wearable frame similar in design to typical frames used to hold spectacle lenses. The frame may include lens mounts or eye-wires 310, 320. Each lens mount 310, 320 may include a conductive coil 25, or a conductive coil may be included only in one frame. The device 300 may be worn in the same manner as a conventional pair of glasses, i.e., by hooking the ends of the arms 330 over the ears of a wearer. The device 300 may be suitable for charging one or more ophthalmic devices implanted within or in the region of a wearer's eye or eyes. The device may include one or more lenses mounted within the mounts 310, 320, such as prescription spectacle lenses, plano lenses, lenses made of glass, plastic, or any other suitable material, or the device may include no lenses at all. The device 300 illustrated in FIGS. 3A-3B may be used, for example, to charge an implanted electronic ophthalmic device without significantly obscuring the vision of the wearer, i.e., when the device is worn the wearer's vision may be substantially or entirely unobscured by the device. In some cases, the device may obscure the wearer's vision about the same as, or no more than, an eyewear frame, such as would typically be used in conjunction with prescription lenses. As a specific example, the device may be worn while the wearer watches television or a monitor, reads, or performs other activities. A power source and control electronics as previously described may be included in a portion of the wearable frame such as the bridge 315, one or both of the mounts 310, 320, or in any other suitable location. Appropriate electrical connections may be disposed within the frame to connect the power source and/or control electronics to the conductive coil 25.

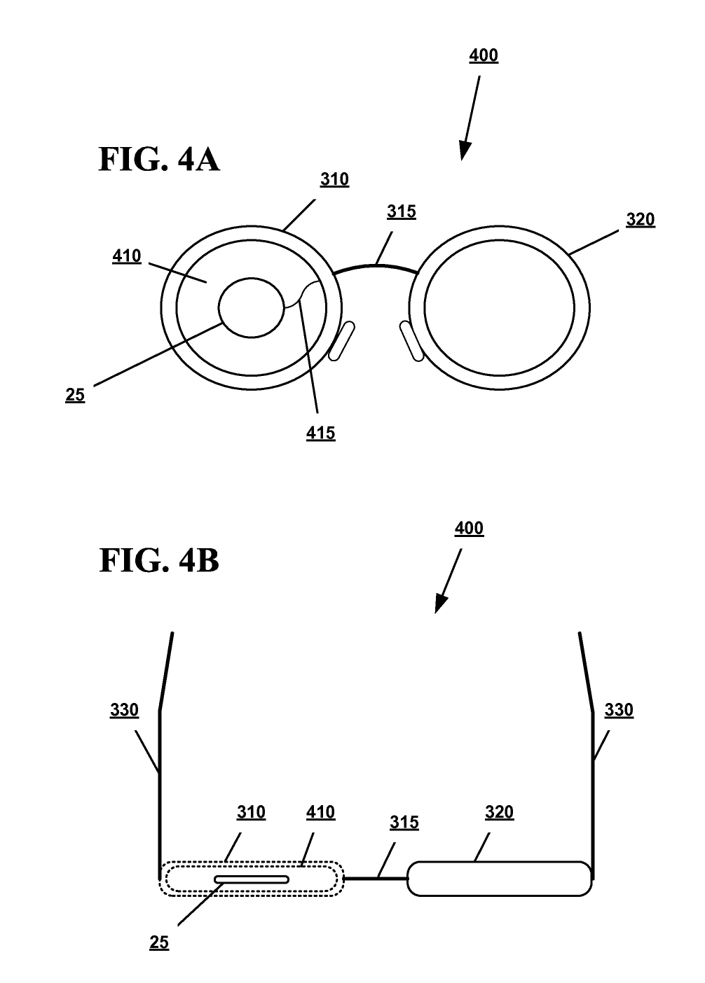

FIGS. 4A-4B show front and top schematic views, respectively, of another device 400 that includes a wearable and a conductive coil for charging one or more implanted ophthalmic devices. The device shown in FIGS. 4A-4B is similar to that shown in FIGS. 3A-3B. However, the conductive coil 25 is disposed within a lens 410 mounted within one of the lens mounts 310. The lens 410 may be a prescription lens, a plano lens, or any other suitable lens type, and may be made of glass, plastic, or any other suitable material. The conductive coil 25 may be disposed within the lens 410, and may have a sufficiently large radius to avoid interfering with the wearer's central vision. An electrical connection 415 may connect the coil 25 to a power source and/or control electronics as previously described. In some configurations, the coil 25 and/or the connection 415 may be sufficiently thin that they are substantially or entirely visible at a close distance, such as when the device 400 is worn by a wearer. In other configurations, the coil and/or the connection may be visible to the wearer's eye, but disposed within the lens so as to leave a central region of the wearer's vision unobstructed.

The devices disclosed herein provide efficient inductive charging devices that are mobile with the wearer. Thus, a wearer may, for example, turn over and sleep in any position, such as when using a mask as illustrated in FIGS. 2A-2B. The devices also may be more user friendly than other charging devices when the user is away from their home.

Any other suitable configurations may be used for the wearable frame, including masks, goggles, hats, headbands, head coverings, adhesive strips, and other arrangements known in the art. Devices for charging implanted electronic devices as described herein may be described as including a conductive coil disposed in or attached to a region of the device. Similarly, an implanted electronic device may be described as being disposed or implanted in a region of the wearer. A charging device may be described as adjacent to a region of the wearer when the device is placed in the vicinity of the region of the wearer. For example, an implanted ophthalmic device may be described as being in the region of the wearer's eye. A conductive coil in a mask or other wearable frame may be described as adjacent to the wearer's eye and/or the implanted device when the wearable frame is worn by the wearer in the usual manner, such as over the eyes (for a mask) or on the face and ears (for a pair of glasses). In general, the conductive coil need not be in direct physical contact with the related region of the wearer to be described as “adjacent to” the region.

Certain configurations of the charging devices described herein may provide different advantages. For example, a mask configuration as described with respect to FIGS. 2A-2B may provide advantages over other configurations, such as those described with respect to FIGS. 3-4. A mask may allow the charging coils in the device to be placed in arbitrary positions relative to the wearer's head and/or an implanted device, whereas a pair of glasses or other similar frame may have a more limited range of positioning options. The mask also may allow a charging coil to be placed closer to the implanted device, which may provide for a more efficient transfer of energy to the implanted device.

While illustrative and presently preferred embodiments of the invention have been described in detail herein, it is to be understood that the inventive concepts may be otherwise variously embodied and employed, and that the appended claims are intended to be construed to include such variations, except as limited by the prior art.

What is claimed is:

1. A device comprising:
a wearable frame; and
a first conductive coil attached to a first region of the wearable frame, the conductive coil disposed adjacent to an eye of a wearer when the wearable frame is worn by the wearer.

2. The device of claim 1, wherein the wearable frame comprises a mask.

3. The device of claim 1, further comprising:
a second conductive coil attached to a second region of the wearable frame;
wherein the first region of the wearable frame is disposed adjacent to one eye of the wearer and the second region of the wearable frame is disposed adjacent to the other eye of the wearer when the frame is worn by the wearer.

4. The device of claim 3, wherein the wearable frame comprises a mask.

5. The device of claim 1, wherein the first conductive coil configured to provide an induction charge to an electronic ophthalmic device implanted within the body of a wearer of the device.

6. The device of claim 1, further comprising a power source electrically connected to the first conductive coil.

7. The device of claim 6, wherein the power source comprises a rechargeable battery.

8. The device of claim 1, wherein the wearable frame comprises one or more lens mounts.

9. The device of claim 8, wherein the conductive coil is disposed within one of the one or more lens mounts.

10. The device of claim 8, wherein the wearable frame comprises a lens disposed within one of the one or more lens mounts.

11. The device of claim 10, wherein the first conductive coil is disposed within the lens.

12. The device of claim 1, wherein the wearer's vision is substantially unobscured when the device is worn.

13. A mask comprising a conductive coil that, when the mask is worn over the eyes of a wearer, is configured to induce an electrical current in a device implanted within the wearer to inductively charge a power source associated with said device.

14. The mask of claim 13, further comprising:
a second conductive coil that, when the mask is worn over the eyes of a wearer, is configured to induce an electrical current in a second device implanted within the wearer to inductively charge a power source associated with said second device.

15. The mask of claim 13, further comprising a power source electrically connected to the first conductive coil.

16. The mask of claim 15, wherein the power source comprises a rechargeable battery.

17. A system comprising:
an implantable ophthalmic device configured to be implanted in a first region of a wearer, the implantable device comprising:
a rechargeable battery; and
a first conductive coil; and
a wearable device comprising:
a wearable frame; and
a second conductive coil attached to a first region of the wearable frame;
wherein, when the wearable device is worn by the wearer, the first region of the wearable frame is disposed adjacent to the first region of the wearer.

18. The system of claim 17, wherein the wearable frame comprises a mask.

19. The system of claim 17, further comprising:
a third conductive coil attached to a second region of the wearable frame.

20. The system of claim 19, further comprising:
a second implantable device configured to be implanted in a second region of the wearer;
wherein, when the wearable device is worn by the wearer, the second region of the wearable frame is disposed adjacent to the second region of the wearer.

21. The system of claim 17, further comprising a power source electrically connected to the first conductive coil.

22. The system of claim 21, wherein the power source comprises a rechargeable battery.

23. The system of claim 17, wherein the wearable frame comprises one or more lens mounts.

24. The system of claim 23, wherein the conductive coil is disposed within one of the one or more lens mounts.

25. The system of claim 23, wherein the wearable frame comprises a lens disposed within one of the one or more lens mounts.

26. The system of claim 25, wherein the first conductive coil is disposed within the lens.

27. The system of claim 23, wherein the wearer's vision is substantially unobscured when the device is worn.

28. A device comprising:
means for attaching the device to a wearer of the device;
means for inductively charging an electronic device implanted within the body of the wearer.

29. The device of claim 28, further comprising:
means for inductively charging a second electronic device implanted within the body of the wearer.

30. The device of claim 1, wherein the wearer's vision is substantially unobscured when the device is worn.
